(19) United States
(12) Patent Application Publication
(10) Pub. No.: US 2010/0041699 A1
(43) Pub. Date: Feb. 18, 2010

(54) MICROCRYSTALLINE (5-FLUORO-2-METHYL-3-QUINOLIN-2-YLMETHYL-INDOL-1-YL) ACETIC ACID

(75) Inventors: Edward Andrew Boyd, Abingdon (GB); Frederick Arthur Brookfield, Abingdon (GB); Christopher James Brennan, Abingdon (GB); Christopher Francis Palmer, Abingdon (GB); Leigh Andre Pearcey, Abingdon (GB); James Matthew Lovell, Abingdon (GB)

Correspondence Address: STERNE, KESSLER, GOLDSTEIN & FOX P.L.L.C., 1100 NEW YORK AVENUE, N.W., WASHINGTON, DC, 20005, US

(73) Assignee: OXAGEN LIMITED, Abingdon, Oxfordshire (GB)

(21) Appl. No.: 11/817,399

(22) PCT Filed: Mar. 1, 2006
(86) PCT No.: PCT/GB2006/000704

§ 371 Date: Feb. 24, 2009

(30) Foreign Application Priority Data
Mar. 1, 2005 (GB) 0504150.4

(51) Int. Cl. A61K 31/47; C07D 215/14; A61P 11/00; A61P 11/02; A61P 11/06

(52) U.S. Cl. 514/314; 546/174

(57) ABSTRACT

The invention relates to a microcrystalline form of a compound which is an inhibitor of PGD₂ at the CRTH2 receptor. The microcrystalline form is obtained from a simple chemical reaction without the need for a milling process.

The present invention relates to a compound which is an inhibitor of PGD₂ at the CRTH2 receptor. In particular, it relates to a microcrystalline form of this compound.

In our earlier patent application No. PCT/GB2004/004417, we describe a number of indole acetic acid derivatives which are inhibitors of PGD₂ at the CRTH2 receptor and which are therefore useful in the treatment or prevention of diseases and conditions such as allergic asthma, perennial allergic rhinitis, seasonal allergic rhinitis, atopic dermatitis, contact hypersensitivity (including contact dermatitis), conjunctivitis, especially allergic conjunctivitis, eosinophilic bronchitis, food allergies, eosinophilic gastroenteritis, inflammatory bowel disease, ulcerative colitis and Crohn's disease, mastocytosis and also other PGD₂-mediated diseases, for example autoimmune diseases such as hyper IgE syndrome and systemic lupus erythematus, psoriasis, acne, multiple sclerosis, allograft rejection, reperfusion injury, chronic obstructive pulmonary disease, as well as, in some cases, rheumatoid arthritis, psoriatic arthritis and osteoarthritis and neurodegenerative diseases such as Alzheimer's disease, Parkinson's disease, stroke and amyoptrophic lateral sclerosis.

It is well known to those of skill in the art that it is often advantageous to prepare microcrystalline forms of pharmaceutically active compounds in order to maximise their surface area which, in turn, maximises their oral absorption by the body from the GI tract. The preparation of such microcrystalline forms usually involves milling the compound to obtain the required particle size and this is, of course, an additional production step which increases the production costs.

Surprisingly, however, the present inventors have found that a microcrystalline form of one of the compounds described in PCT/GB2004/004417 can be prepared simply and inexpensively without additional process steps.

Therefore, in a first aspect of the present invention there is provided a microcrystalline form of (5-fluoro-2-methyl-3-quinolin-2-ylmethyl-indol-1-yl)-acetic acid, wherein at least 90% of the crystals have a diameter not greater than about 3 μm.

It is preferred that at least 90% of the crystals have a diameter not greater than about 2 μm and particularly preferred that at least 90% of the crystals have a diameter not greater than about 1 μm.

Surprisingly, it has been found that the microcrystalline form of this compound can be prepared by a simple and inexpensive route which does not involve a milling process.

In our earlier application, we described the preparation of compounds such as (5-fluoro-2-methyl-3-quinolin-2-ylmethyl-indol-1-yl)-acetic acid from their ethyl esters by hydrolysis using lithium hydroxide monohydrate in a 1:1 mixture of tetrahydrofuran and water. When the product was recrystallised from dimethylsulfoxide/water (DMSO/water), it was found that the diameter of 90% of the crystals was less than about 50-70 μm.

However, when the DMSO was removed from the recrystallised product by treating with a mild aqueous base followed by citric acid, it was surprisingly found that the product was obtained in the form of a microcrystalline solid having a crystal diameter of less than 5 μm and, in fact, generally about 1 μm or less.

Therefore, in a second aspect of the invention, there is provided a process for the preparation of a microcrystalline form of (5-fluoro-2-methyl-3-quinolin-2-ylmethyl-indol-1-yl)-acetic acid, wherein at least 90% of the crystals have a diameter not greater than about 3 μm, the process comprising:

i. treating crystalline (5-fluoro-2-methyl-3-quinolin-2-ylmethyl-indol-1-yl)-acetic acid with an aqueous base; and
ii. treating with a weak acid; and
iii. collecting the precipitated microcrystalline (5-fluoro-2-methyl-3-quinolin-2-ylmethyl-indol-1-yl)-acetic acid.

Suitable bases for use in the method of the invention have a pKb greater than 5.5 and include, for example, carbonates, for example sodium, potassium or ammonium carbonate. Potassium carbonate is particularly useful.

In step (i) of the method, the mixture of the crystalline solid and the weak base may be heated to obtain partial dissolution of the (5-fluoro-2-methyl-3-quinolin-2-ylmethyl-indol-1-yl)-acetic acid. When the weak base is a carbonate such as potassium carbonate, heating to about 45 to 60° C., and preferably 50 to 55° C., has been found to be appropriate.

The term “weak acid” as used in step (ii) of the method is a term known in the art, and means an acid that partially dissociates in an aqueous solution. In the context of the present invention, a weak acid is an acid having a pKa of 2 or more such that it is able to precipitate (5-fluoro-2-methyl-3-quinolin-2-ylmethyl-indol-1-yl)-acetic acid, which has a pKa of value of 2.8.

Suitable weak acids for use in step (ii) include citric acid, tartaric acid and benzene sulfonic acid with citric acid being particularly suitable.

In step (ii), the amount of weak acid is chosen to adjust the pH of the solution to less than about pH 6, and more typically to about pH 5.5 to ensure that the acid precipitates from the solution.

It is preferred to add the acid slowly over a period of about 1 to 5 hours and to cool the solution, for example to about 10 to 30° C., preferably 15 to 25° C., during the addition of the acid.

Step (i) of the process set out above may be preceded by one or more of the steps of:

a. Hydrolysing a C₁-C₆ alkyl ester of (5-fluoro-2-methyl-3-quinolin-2-ylmethyl-indol-1-yl)-acetic acid with a base to give (5-fluoro-2-methyl-3-quinolin-2-ylmethyl-indol-1-yl)-acetic acid; and
b. recrystallising the (5-fluoro-2-methyl-3-quinolin-2-ylmethyl-indol-1-yl)-acetic acid from a polar organic solvent.

Typically, the base used in step (a) is an alkali metal hydroxide such as lithium, sodium or potassium hydroxide in a mixture of water and an organic solvent such as tetrahydrofuran (THF).

The product (5-fluoro-2-methyl-3-quinolin-2-ylmethyl-indol-1-yl)-acetic acid is sparingly soluble in most solvents but DMSO, N-methylpyrrolidine and dimethylformamide, any of which may optionally be mixed with water, are all suitable solvents for the recrystallisation step, with a mixture of DMSO and water being particularly preferred.

As mentioned above, the microcrystalline (5-fluoro-2-methyl-3-quinolin-2-ylmethyl-indol-1-yl)-acetic acid is an antagonist of PGD₂ at the CRTH2 receptor and is therefore a useful method for the treatment of diseases and conditions mediated by PGD₂ at the CRTH2 receptor, the method comprising administering to a patient in need of such treatment a suitable amount of microcrystalline (5-fluoro-2-methyl-3-quinolin-2-ylmethyl-indol-1-yl)-acetic acid.

In a further aspect of the invention, there is provided microcrystalline form of (5-fluoro-2-methyl-3-quinolin-2-ylmethyl-indol-1-yl)-acetic acid, wherein at least 90% of the crystals have a diameter not greater than about 3 μm, for use in medicine, particularly for use in the treatment or prevention of diseases and conditions mediated by PGD₂ at the CRTH2 receptor.

As mentioned above, such diseases and conditions include allergic asthma, perennial allergic rhinitis, seasonal allergic rhinitis, atopic dermatitis, contact hypersensitivity (including contact dermatitis), conjunctivitis, especially allergic conjunctivitis, eosinophilic bronchitis, food allergies, eosinophilic gastroenteritis, inflammatory bowel disease, ulcerative colitis and Crohn's disease, mastocytosis and also other PGD₂-mediated diseases, for example autoimmune diseases such as hyper IgE syndrome and systemic lupus erythematus, psoriasis, acne, multiple sclerosis, allograft rejection, reperfusion injury, chronic obstructive pulmonary disease, as well as rheumatoid arthritis, psoriatic arthritis and osteoarthritis and neurodegenerative diseases such as Alzheimer's disease, Parkinson's disease, stroke and amyoptrophic lateral sclerosis.

The microcrystalline (5-fluoro-2-methyl-3-quinolin-2-ylmethyl-indol-1-yl)-acetic acid must be formulated in an appropriate manner depending upon the diseases or conditions it is required to treat.

Therefore, in a further aspect of the invention there is provided a pharmaceutical composition comprising a microcrystalline form of (5-fluoro-2-methyl-3-quinolin-2-ylmethyl-indol-1-yl)-acetic acid, wherein the crystals have a diameter not greater than about 3 μm, together with a pharmaceutical excipient or carrier. Other active materials may also be present, as may be considered appropriate or advisable for the disease or condition being treated or prevented.

The carrier, or, if more than one be present, each of the carriers, must be acceptable in the sense of being compatible with the other ingredients of the formulation and not deleterious to the recipient.

The formulations include those suitable for oral, rectal, nasal, bronchial (inhaled), topical (including eye drops, buccal and sublingual), vaginal or parenteral (including subcutaneous, intramuscular, intravenous and intradermal) administration and may be prepared by any methods well known in the art of pharmacy.

The route of administration will depend upon the condition to be treated but preferred compositions are formulated for oral, nasal, bronchial or topical administration.

The composition may be prepared by bringing into association the above defined active agent with the carrier. In general, the formulations are prepared by uniformly and intimately bringing into association the active agent with liquid carriers or finely divided solid carriers or both, and then if necessary shaping the product. The invention extends to methods for preparing a pharmaceutical composition comprising bringing microcrystalline (5-fluoro-2-methyl-3-quinolin-2-ylmethyl-indol-1-yl)-acetic acid, wherein the crystals have a diameter not greater than about 3 μm, in conjunction or association with a pharmaceutically or veterinarily acceptable carrier or vehicle.

Formulations for oral administration in the present invention may be presented as; discrete units such as capsules, sachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

For topical application to the skin, microcrystalline (5-fluoro-2-methyl-3-quinolin-2-ylmethyl-indol-1-yl)-acetic acid may be made up into a cream, ointment, jelly, solution or suspension etc. Cream or ointment formulations that may be used for the drug are conventional formulations well known in the art, for example, as described in standard text books of pharmaceutics such as the British Pharmacopoeia.

Microcrystalline (5-fluoro-2-methyl-3-quinolin-2-ylmethyl-indol-1-yl)-acetic acid may be used for the treatment of the respiratory tract by nasal, bronchial or buccal administration of, for example, aerosols or sprays which can disperse the pharmacological active ingredient in the form of a powder or in the form of drops of a solution or suspension. Pharmaceutical compositions with powder-dispersing properties usually contain, in addition to the active ingredient, a liquid propellant with a boiling point below room temperature and, if desired, adjuncts, such as liquid or solid non-ionic or anionic surfactants and/or diluents. Pharmaceutical compositions in which the pharmacological active ingredient is in solution contain, in addition to this, a suitable propellant, and furthermore, if necessary, an additional solvent and/or a stabiliser. Instead of the propellant, compressed air can also be used, it being possible for this to be produced as required by means of a suitable compression and expansion device.

Parenteral formulations will generally be sterile.

Typically, the dose of the microcrystalline (5-fluoro-2-methyl-3-quinolin-2-ylmethyl-indol-1-yl)-acetic acid will be about 0.01 to 100 mg/kg; so as to maintain the concentration of drug in the plasma at a concentration effective to inhibit PGD₂ at the CRTH2 receptor. The precise amount of microcrystalline (5-fluoro-2-methyl-3-quinolin-2-ylmethyl-indol-1-yl)-acetic acid which is therapeutically effective, and the route by which such compound is best administered, is readily determined by one of ordinary skill in the art by comparing the blood level of the agent to the concentration required to have a therapeutic effect.

Microcrystalline (5-fluoro-2-methyl-3-quinolin-2-ylmethyl-indol-1-yl)-acetic acid may be used in combination with one or more active agents which are useful in the treatment of the diseases and conditions listed above, although these active agents are not necessarily inhibitors of PGD₂ at the CRTH2 receptor.

Therefore, the pharmaceutical composition described above may additionally contain one or more of these active agents.

There is also provided the use of microcrystalline (5-fluoro-2-methyl-3-quinolin-2-ylmethyl-indol-1-yl)-acetic acid, wherein the crystals have a diameter not greater than about 3 μm, in the preparation of an agent for the treatment of diseases and conditions mediated by PGD₂ at the CRTH2 receptor, wherein the agent also comprises an additional active agent useful for the treatment of the same diseases and conditions.

These additional active agents which may have a completely different mode of action include existing therapies for allergic and other inflammatory diseases including:

β2 agonists such as salmeterol;
corticosteroids such as fluticasone;
antihistamines such as loratidiine;
leukotriene antagonists such as montelukast;
anti-IgE antibody therapies such as omalizumab;
anti-infectives such as fusidic acid (particularly for the treatment of atopic dermatitis);
anti-fungals such as clotrimazole (particularly for the treatment of atopic dermatitis);
immunosuppressants such as tacrolimuts and particularly pimecrolimus in the case of inflammatory skin disease.

CRTH2 antagonists may also be combined with therapies that are in development for inflammatory indications including:

other antagonists of PGD₂ acting at other receptors, such as DP antagonists;
inhibitors of phosphodiesterase type 4 such as cilonilast;
drugs that modulate cytokine production such as inhibitors of TNFα converting enzyme (TACE);
drugs that modulate the activity of Th2 cytokines IL-4 and IL-5 such as blocking monoclonal antibodies and soluble receptors;
PPAR-γ agonists such as rosiglitazone;
5-lipoxygenase inhibitors such as zileuton.

In yet a further aspect of the invention, there is provided a product comprising microcrystalline (5-fluoro-2-methyl-3-quinolin-2-ylmethyl-indol-1-yl)-acetic acid, wherein the crystals have a diameter not greater than about 3 μm, and one or more of the agents listed above as a combined preparation for simultaneous, separate or sequential use in the treatment of a disease or condition mediated by the action of PGD₂ at the CRTH2 receptor.

The invention will now be described in greater detail with reference to the following examples.

### EXAMPLE 1

### Synthesis of microcrystalline (5-fluoro-2-methyl-3-quinolin-2-ylmethyl-indol-1-yl)-acetic acid

The synthesis was conducted according to the reaction scheme set out in Scheme 1.

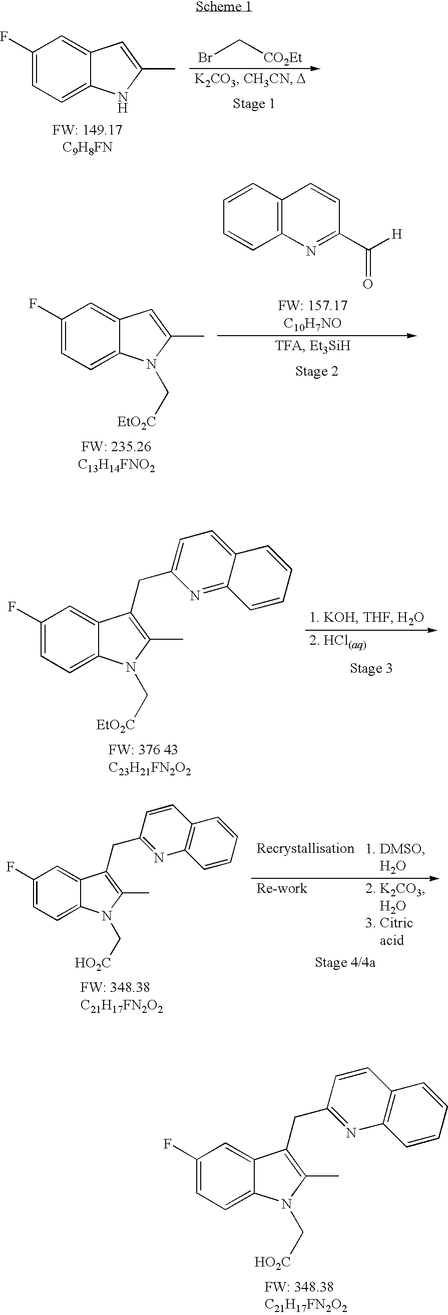

### Stage 1: Synthesis of ethyl-(5-fluoro-2-methylindolyl-1-acetate)

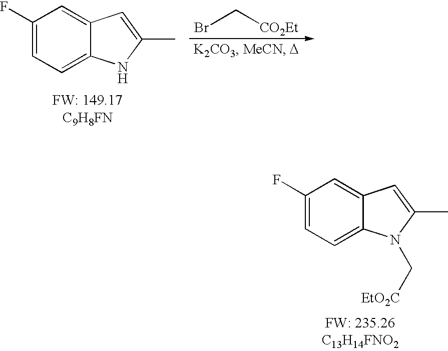

5-Fluoro-2-methylindole (0.45 Kg, 3.017 mol, 1.0 wt), powdered potassium carbonate (1.251 Kg, 9.05 mol, 2.78 wt) and acetonitrile (9.0 L, 20 vol) were charged to a 20 L flange flask at 15 to 25° C. Ethyl bromoacetate (0.671 L, 2.67 mol, 1.49 vol) was added and the resulting suspension heated to and maintained at reflux for 18 h after which time in-process check analysis by ¹H NMR¹ indicated 87% conversion. A further charge of ethyl bromoacetate (0.333 L, 1.32 mol, 0.74 vol) and powdered potassium carbonate (0.626 Kg, 4.53 mol, 1.39 wt) was made and reflux conditions established for a further 6 hours. In-process check by ¹H NMR¹ analysis indicated 98.4% conversion. The flask contents were allowed to cool to 15 to 25° C. over 16 hours. The solids were removed by filtration and the filter-cake washed with acetonitrile (2×1 L, 2×2 vol). The combined filtrates were concentrated to dryness under vacuum at up to 40° C. (water bath) to provide crude Stage 1 as a brown oil (1.286 Kg). The crude product was purified by dry flash chromatography using a gradient elution from heptanes to heptanes:toluene to toluene to give ethyl-(5-fluoro-2-methylindolyl-1-acetate) as an off-white solid (0.573 Kg, 80.7% theoretical, corrected for residual toluene). Mixed fractions were re-chromatographed as appropriate. ¹Reaction sampled, the sample concentrated, the residue taken up in D₆-DMSO, filtered and the ¹H NMR spectrum recorded

### Stage 2: Synthesis of (5-Fluoro-2-methyl-3-quinolin-2-ylmethylindo-1-yl)-acetic acid ethyl ester

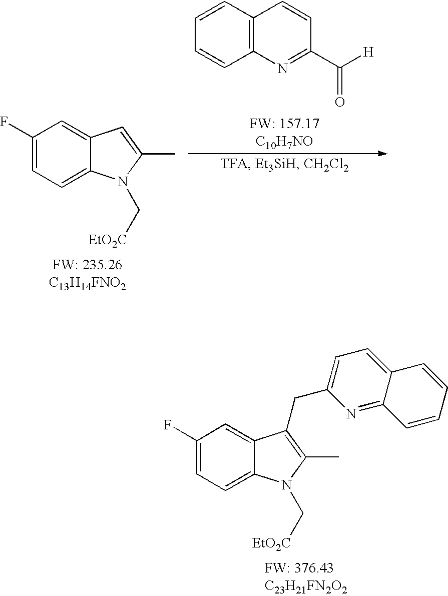

Ethyl-(5-fluoro-2-methylindolyl-1-acetate) (0.573 Kg, 2.44 mol, 4.0 wt) and quinoline-2-carboxaldehyde (0.418 Kg, 2.66 mol, 0.735 wt) as a solution in dichloromethane (5.73 L, 10 vol) at 0 to 5° C. were treated with triethylsilane (1.369 L, 8.51 mol, 2.39 vol) followed by the drop-wise addition of trifluoroacetic acid (0.561 L, 7.28 mol, 0.98 vol) at 0 to 10° C. The resulting dark red solution was warmed to and maintained at reflux for 3 h after which time in-process check analysis by ¹H NMR² indicated reaction completion. The reaction was cooled to 15 to 25° C. and quenched by the addition of saturated sodium hydrogen carbonate solution (11.5 L, 20 vol) over 0.5 h (note: foaming and gas evolution). The layers were separated, the aqueous layer extracted with dichloromethane (1×2.8 L, 1×5.0 vol), the combined organics washed with 20% w/w aqueous sodium chloride solution (1×3.0 L, 1×5 vol) and dried over sodium sulfate (0.6 Kg, 1.05 wt). The suspension was filtered, the filter-cake washed with dichloromethane (2×0.6 L, 2×1.05 vol) and the combined filtrates concentrated under vacuum at up to 40° C. (water bath) to afford (5-fluoro-2-methyl-3-quinolin-2-ylmethylindo-1-yl)-acetic acid ethyl ester as a brown oily solid (1.227 Kg, 133.8% theoretical) contaminated with silyl-related by-products. ² MET/PR/0344

### Stage 3: (5-Fluoro-2-methyl-3-quinolin-2-ylmethylindo-1-yl)-acetic acid

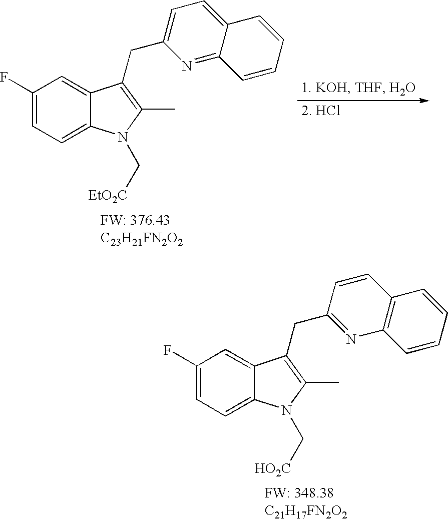

For the purposes of the Stage 3 input calculations, it was assumed that the Stage 2 reaction had progressed in 100% theoretical yield.

Potassium hydroxide (0.486 Kg, 0.53 wt) as a solution in water (5.5 L, 6 vol) was added to a solution of (5-fluoro-2-methyl-3-quinolin-2-ylmethyl-indo-1-y)l-acetic acid ethyl ester (0.916 Kg assumed, 2.44 mol, 1 wt) in tetrahydrofuran (3.66 L, 4 vol) such that the reaction mixture was allowed to exotherm to 30 to 35° C. The reaction was maintained at 30 to 35° C. for 2 h after which time TLC³ analysis (ethyl acetate:toluene 1:1; visualisation: LTV) indicated reaction completion by the absence of starting material. tert-Butyl methyl ether (4.6 L, 5 vol) was added and the phases separated such that interfacial material was retained with the aqueous phase. The aqueous layer was washed further with tert-butyl methyl ether (4.6 L, 5 vol), concentrated under vacuum at 35 to 40° C. (water bath) for up to 1 h to remove residual organics and then cooled to 15 to 25° C. The resulting slurry was acidified with aqueous hydrochloric acid (2M, 3.44 L, 3.75 vol) to pH 5.5 such that the temperature was maintained in the range 20 to 25° C. (noted that the solution turned a deep red colour on acidification). The slurry was aged for 1 hour at 15 to 25° C., the pH confirmed as 5.5, the slurry filtered (slow) and the collected solids washed with water (1×1 vol, 1×0.92 L). The wet-cake was azeo-dried with toluene (35 L) until the water content was 0.3% by Karl Fisher analysis affording the crude product as a purple solid (0.767 Kg, 90.5% theoretical corrected for 5.6% w/w toluene). ³ Reaction mixture diluted with THF:water prior to analysis

### Stage 4/4a: Recrystallisation and reprecipitation of (5-fluoro-2-methyl-3-quinolin-2-ylmethylindo-1-yl)-acetic acid

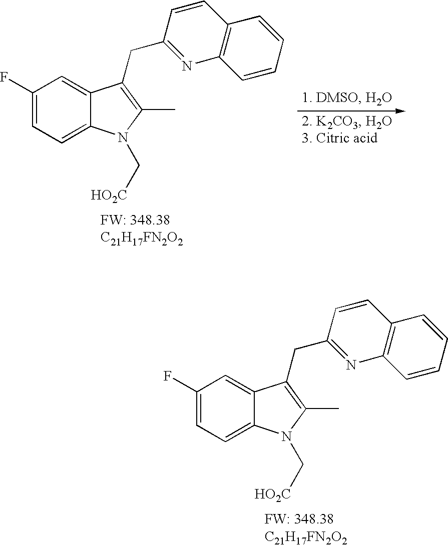

A slurry of (5-fluoro-2-methyl-3-quinolin-2-ylmethylindo-1-yl)-acetic acid (0.767 Kg, 2.2 mol, 1.0 wt) in dimethyl sulfoxide (9.21 L, 12 vol) was heated to 95 to 100° C. to effect dissolution. The resultant was hot filtered at 95° C., the filtrates treated with water (2.3 L, 3.0 vol) over 10 minutes such that the temperature was maintained in the range 70 to 80° C. and cooled to 15 to 25° C. over 3 hours. The observed precipitate was collected by filtration, the collected yellow solids washed with water (3×0.8 L, 3×1 vol), pulled dry on the filter and blended with a further 0.175 Kg of (5-fluoro-2-methyl-3-quinolin-2-ylmethylindo-1-yl)-acetic acid from an earlier batch. The blended material was dried under vacuum at up to 45° C. for 16 hours (0.942 Kg). ¹H NMR analysis (D₆-DMSO) indicated the presence of 0.6% w/w dimethyl sulfoxide.

A slurry of (5-fluoro-2-methyl-3-quinolin-2-ylmethylindo-1-yl)-acetic acid (0.942 Kg, 2.28 mol, 1.0 wt) and potassium carbonate (0.953 Kg, 1.20 wt) in water (12.71 L, 12 vol) was heated to 50 to 55° C. and stirred for 40 minutes to obtain partial dissolution of the solids. Aqueous citric acid (20% w/v) was added over 3 h to adjust the pH to 5.5 (6.54 L, 8.23 vol) with cooling to 15 to 25° C. (note: foaming). Stirring was continued for 0.5 h, the pH confirmed as 5.5 and the observed precipitate collected by filtration (slow). The collected solids were washed with water (2×2.78 L, 2×3.5 vol), pulled dry on the filter, further dried under vacuum to constant weight at up to 45° C. and sieved through a 1.4 mm mesh to give (5-fluoro-2-methyl-3-quinolin-2-ylmethylindo-1-yl)-acetic acid as a yellow solid (0.722 Kg).

### EXAMPLE 2

### Crystal Sizes

The sizes of the crystals of one batch of Product A ((5-fluoro-2-methyl-3-quinolin-2-ylmethylindo-1-yl)-acetic acid recrystallised from DMSO/water as described above in Example 1, step 4) and three batches of Product B ((5-fluoro-2-methyl-3-quinolin-2-ylmethylindo-1-yl)-acetic acid recrystallised from DMSO/water and then treated with potassium carbonate and citric acid as described above in Example 1, step 4a) were measured by laser diffraction and compared. The results are set out below in Table 1.

TABLE 1

| Product | % Particles under given diameter | Particle Diameter (μm) |
|---|---|---|
| A | 90 | 52.13 |
| | 50 | 30.13 |
| | 10 | 11.93 |
| B (Batch 1) | 90 | 0.92 |
| | 50 | 0.27 |
| | 10 | 0.10 |
| B (Batch 2) | 90 | 0.80 |
| | 50 | 0.37 |
| | 10 | 0.20 |
| B (Batch 3) | 90 | 1.53 |
| | 50 | 0.31 |
| | 10 | 0.11 |

It can be seen from the results set out in Table 1 that while only 10% of the particles from product A are less than about 10 μm in diameter, 90% of the particles of product B are less than 2 μm in diameter.

This means that a pharmaceutical formulation containing the microcrystalline product B of the present invention will have significantly improved oral absorption into the body when compared with the product A, which is the product disclosed in our earlier application.

1. A microcrystalline form of (5-fluoro-2-methyl-3-quinolin-2-ylmethyl-indol-1-yl)-acetic acid, wherein at least 90% of the crystals have a diameter not greater than about 3 μm.

2. A microcrystalline form of (5-fluoro-2-methyl-3-quinolin-2-ylmethyl-indol-1-yl)-acetic acid, wherein at least 90% of the crystals have a diameter not greater than about 2 μm.

3. A process for the preparation of a microcrystalline form of (5-fluoro-2-methyl-3-quinolin-2-ylmethyl-indol-1-yl)-acetic acid as claimed in claim 1, the process comprising:
i. treating crystalline (5-fluoro-2-methyl-3-quinolin-2-ylmethyl-indol-1-yl)-acetic acid with an aqueous weak base; and
ii. treating with a weak acid; and
iii. collecting the precipitated microcrystalline (5-fluoro-2-methyl-3-quinolin-2-ylmethyl-indol-1-yl)-acetic acid.

4. A process as claimed in claim 3, wherein the weak base is sodium carbonate, potassium carbonate or ammonium carbonate.

5. A process as claimed in claim 4, wherein the weak base is potassium carbonate.

6. A process as claimed in claim 3, wherein in step (i), the mixture of the crystalline solid and the weak base is heated to obtain partial dissolution of the (5-fluoro-2-methyl-3-quinolin-2-ylmethyl-indol-1-yl)-acetic acid.

7. A process as claimed in claim 6, wherein the weak base is potassium carbonate and the mixture is heated to 50 to 55° C.

8. A process as claimed in claim 3, wherein the weak acid is citric acid, tartaric acid or benzene sulfonic acid.

9. A process as claimed in claim 8, wherein the weak acid is citric acid.

10. A process as claimed in claim 3, wherein step (i) of the process is preceded by one or more of the steps of:
a. hydrolysing a C₁-C₆ alkyl ester of (5-fluoro-2-methyl-3-quinolin-2-ylmethyl-indol-1-yl)-acetic acid with a base to give (5-fluoro-2-methyl-3-quinolin-2-ylmethylindol-1-yl)-acetic acid; and
b. recrystallising the (5-fluoro-2-methyl-3-quinolin-2-ylmethyl-indol-1-yl)-acetic acid from a polar organic solvent.

11. A process as claimed in claim 10 wherein, in step (a) the base is an alkali metal hydroxide in a mixture of water and an organic solvent.

12. A process as claimed in claim 10, wherein the polar organic solvent of step (b) is DMSO, N-methylpyrrolidine or dimethylformamide, any of which may optionally be mixed with water.

13. A method for treating a subject suffering from or at risk for acquiring a PGD₂-mediated disease comprising administering to the subject an amount effective to inhibit PGD₂ in the subject of a microcrystalline form of (5-fluoro-2-methyl-3-quinolin-2-ylmethyl-indol-1-yl)-acetic acid, wherein at least 90% of the crystals have a diameter not greater than about 3 μm.

14. A method of claim 13 wherein the PGD₂-mediated disease is selected from the group consisting of allergic asthma, perennial allergic rhinitis, seasonal allergic rhinitis, atopic dermatitis, contact hypersensitivity (including contact dermatitis), conjunctivitis, eosinophilic bronchitis, food allergies, eosinophilic gastroenteritis, inflammatory bowel disease, ulcerative colitis and Crohn's disease, mastocytosis, autoimmune diseases and neurodegenerative diseases.

15. (canceled)

16. A pharmaceutical composition comprising a microcrystalline form of (5-fluoro-2-methyl-3-quinolin-2-ylmethyl-indol-1-yl)-acetic acid, as claimed in claim 1, together with a pharmaceutical excipient or carrier.

17. A pharmaceutical composition as claimed in claim 16 formulated for oral, nasal, bronchial or topical administration.

18. A pharmaceutical composition as claimed in claim 16, further comprising one or more additional active agents useful in the treatment of diseases and conditions mediated by PGD₂ at the CRTH2 receptor.

19. A pharmaceutical composition as claimed in claim 18, wherein the additional active agents are selected from:
β agonists;
corticosteroids;
antihistamines;
leukotriene antagonists;
anti-IgE antibody therapies;
anti-infectives;
anti-fungals;
immunosuppressants;
antagonists of PGD2 acting at receptors other than CRTH2;
inhibitors of phosphodiesterase type 4;
drugs that modulate cytokine production;
drugs that modulate the activity of Th2 cytokines IL-4 and IL-5;
PPAR-γ agonists; and
5-lipoxygenase inhibitors.

20. A process for the preparation of a pharmaceutical composition as claimed in claim 16, the process comprising bringing a compound as claimed in claim 1 in conjunction or association with a pharmaceutically or veterinarily acceptable carrier or vehicle.

21. (canceled)

22. The method of claim 13 which further comprises simultaneously, separately or sequentially administering to the subject one or more additional active agents useful for the treatment of diseases and conditions mediated by PGD2 at the CRTH2 and/or DP receptor.

23. The method as claimed in claim 22, wherein the additional active agents are selected from the group consisting of β agonists, corticosteroids, antihistamines, leukotriene antagonists, anti-IgE antibody therapies, anti-infectives, anti-fungals, immunosuppressants, antagonists of PGD2 acting at other than CRTH2, inhibitors of phosphodiesterase type 4, drugs that modulate cytokine production, drugs that modulate the activity of Th2 cytokines IL-4 and IL-5. PPAR-γ agonists and 5-lipoxygenase inhibitors.

24. The process of claim 11, wherein the base is lithium, sodium or potassium hydroxide in a mixture of water and tetrahydrofuran (THF).

25. The method of claim 14, wherein the PGD2-mediated disease is an autoimmune disease selected from the group consisting of hyper IgE syndrome, systemic lupus erythematus, psoriasis, acne, multiple sclerosis, allograft rejection, reperfusion injury, chronic obstructive pulmonary disease, rheumatoid arthritis, psoriatic arthritis and osteoarthritis.

26. The method of claim 14, wherein the PGD2-mediated disease is an neurodegenerative disease selected from the group consisting of Alzheimer's disease, Parkinson's disease, stroke and amyoptrophic lateral sclerosis.

27. A pharmaceutical composition as claimed in claim 18, wherein the additional active agents are selected from salmeterol, fluticasone, loratidine, montelukast, omalizumab, fusidic acid, clotrimazole, tacrolimus, pimecrolimus, DP antagonists, cilonilast, inhibitors of TNFα converting enzyme (TACE), monoclonal antibodies and soluble receptors that modulate the activity of Th2 cytokines IL-4 and IL-5, rosiglitazone and zileuton.
